(19) United States
(12) Patent Application Publication
(10) Pub. No.: US 2023/0047991 A1
(43) Pub. Date: Feb. 16, 2023

(54) ISOVOLUMETRIC PUMP AND SYSTEMS AND METHODS THEREOF

(71) Applicant: Washington University, St. Louis, MO (US)

(72) Inventors: GUY GENIN, St. Louis, MO (US); JOHN CASHIN, St. Louis, MO (US); MOHAMED ZAYED, St. Louis, MO (US); SANGHUN LEE, St. Louis, MO (US)

(21) Appl. No.: 17/796,035

(22) PCT Filed: Jan. 29, 2021
(86) PCT No.: PCT/US2021/015908

§ 371 Date: Jul. 28, 2022

Related U.S. Application Data
(60) Provisional application No. 62/968,619, filed on Jan. 31, 2020.

(51) Int. Cl. A61M 5/168

(52) U.S. Cl. CPC A61M 5/16886; A61M 5/16854; A61M 2205/3334

(57) ABSTRACT

The disclosure provides for an isovolumetric pump system for the removal of a thrombus and a method of use thereof. The isovolumetric pump system includes at least one inflow container connected to an output of the treatment region, at least one outflow container connected to an input of the treatment region, and a drawbar connecting the inflow container and the outflow container. As the drawbar is drawn back, the same quantity of fluid is drawn from the treatment region into the inflow container and injected into the treatment region from the outflow container.

### CROSS-REFERENCE TO RELATED APPLICATIONS

This application claims priority under 35 USC § 119(e) to U.S. Patent Application Ser. No. 62/968,619, filed on Jan. 31, 2020, the entire contents of which are hereby incorporated by reference.

### FIELD

The present disclosure is directed to an isovolumetric pump and systems and methods of use thereof.

### BACKGROUND

Venous thromboembolism (VTE) occurs when a clot (thrombus) or deep venous thrombosis (DVT) breaks off and travels upstream in the venous circulation. A DVT develops from venous stasis, injury, hypercoagulability, or systemic inflammation (as observed with infections like COVID19). Individuals with VTE and COVID19 have mortality rates that can reach up to 85%. Prevention of VTE-related complications in high-risk individuals requires effective thrombus removal (thrombectomy) without harming the veins.

Mechanical thrombectomy devices fragment intraluminal venous thrombus and risk distal embolization of the thrombus fragments unless the fragments are removed. Suction thrombectomy devices apply negative retrograde pressure leading to removal of intraluminal thrombus fragments but in the process will lead to loss of blood and can lead to rapid collapse of the target vessel. Rapid collapse of the target vessel may lead to damage to the vessel wall. A method that can flush thrombus fragments from the target vessel can help evacuate the lumen, reduce the risk of distal embolization, and minimize blood loss. However flushing of the target vessel segment may also lead to excessive distention of the target vessel. Excessive distention of the target vessel may lead to damage of the vessel wall.

Similarly, arterial atherectomy/thrombectomy requires removal of arterial intraluminal material. In segments of arterial occlusion, removal of this luminal material can induce rapid collapse of the arterial wall due to vacuum induced in the lumen. This can cause damage to the arterial wall and lumen. Infusion of thrombolytic medications in the vessel segment can also induce damage to the arterial wall and lumen by over-distension. Isovolumetric infusion and suction in a thrombosed/occluded arterial segment would help reduce the risk of arterial wall collapse and/or distension during removal of intraluminal material.

Therefore, there is a need for a device that can maintain isovolumetric inflow and outflow in the target vessel which can facilitate flushing with reduced risk of vessel collapse or distention. Reduced risk of vessel collapse or distention would reduce the risk of vessel wall damage.

### BRIEF SUMMARY

The disclosure provides for a device that exchanges equal continuous inflow and outflow fluid volumes. An isovolumetric pump system for removal of a thrombus from a treatment region may include at least one inflow container connected to an output of the treatment region, at least one outflow container connected to an input of the treatment region, and a drawbar connecting the inflow container and the outflow container. As the drawbar is drawn back, the same quantity of fluid is drawn from the treatment region into the inflow container and injected into the treatment region from the outflow container. The treatment region may be a region within a vessel containing a thrombus and the isovolumetric pump system may be operable to remove the thrombus from the treatment region. The isovolumetric pump is operable to ensure that the walls of the vessel neither distend nor collapse while internal volume is exchanged in the treatment region.

In some aspects, the at least one inflow container and the at least one outflow container are syringes. The at least one inflow container may comprise at least 3 inflow syringes and the at least one outflow container may comprise at least 3 outflow syringes. The inflow syringes may be connected using a branch and the outflow syringes may be connected using a branch. In some aspects, the isovolumetric pump system further includes a catheter system connected to the at least one inflow container and the at least one outflow container through tubing, where the catheter system is operable to be deployed within the treatment region and isolate the treatment region. In additional aspects, the isovolumetric pump system may further include a motorized screw and/or at least one vacuum gauge connected to the at least one inflow container, the at least one outflow container, and/or the drawbar. The motorized screw may be controlled by an operator using a rheostat or may be controlled automatically. The vacuum gauges may be operable to ensure that a blockage within the isovolumetric pump system is addressed prior to it affecting treatment.

Further disclosed herein is an isovolumetric pump system for removal of a thrombus from a treatment region that includes an infusion pump connected to an input of the treatment region, the infusion pump operable to pump an infusion fluid into the treatment region; a suction pump connected to an output of the treatment region, the suction pump operable to pump fluid out of the treatment region; at least one motor drive operatively connected to the infusion pump and the suction pump; and a controller operatively connected to the infusion pump and the suction pump via the at least one motor drive. The controller is operable to ensure that substantially the same quantity of fluid is drawn from the treatment region by the suction pump and injected into the treatment region by the infusion pump. In some aspects, the one or more motor drives is a dual motor drive.

The treatment region is a region within a vessel containing a thrombus and the isovolumetric pump system is operable to remove the thrombus from the treatment region. In some aspects, the isovolumetric pump system further includes a catheter system operable to be placed within the vessel and isolate the treatment region. The catheter system may be connected to the infusion pump and the suction pump through tubing. The isovolumetric pump may be operable to ensure that the walls of the vessel neither distend nor collapse while internal volume is exchanged in the treatment region.

In some aspects, the isovolumetric pump system further includes a pressure sensor connected to the infusion pump, the suction pump, and/or the controller. The pressure sensor and the controller may be operable to perform pressure compensation within the treatment region and control the infusion pump and the suction pump automatically.

In additional aspects, the isovolumetric pump system further includes a housing that has at least one compartment for containing the infusion pump, the suction pump, the one or more motor drives, and the controller; two or more openings on a side of the housing operable for receiving tubing connected to the infusion pump and the suction pump; and an outer surface operable for supporting a user interface. The user interface may include an LCD or analog screen connected to the controller and operable for displaying inflow and outflow rates from the infusion pump and the suction pump; a speed knob switch operatively connected to the one or more motor drives; and an on/off switch.

Also disclosed herein is a method of removing of a thrombus from a treatment region. This process is conducted by linking the means of inflow and outflow to one another, and performing the exchange through manual or electronically automated operation. In an aspect, the method may include connecting at least one inflow container of an isovolumetric pump system to an output of the treatment region, connecting at least one outflow container of the isovolumetric pump system to an input of the treatment region, withdrawing a drawbar connected to the at least one inflow container and the at least one outflow container. The same quantity of fluid is drawn from the treatment region into the inflow container and injected into the treatment region from the outflow container.

The treatment region may be a region within a vessel containing a thrombus and the isovolumetric pump system may be operable to remove the thrombus from the treatment region. The method may further include pulling fluid and the thrombus from the treatment region into the at least one inflow container and simultaneously pushing fluid from the at least one outflow container into the treatment region to ensure that the walls of the vessel neither distend nor collapse while internal volume is exchanged in the treatment region. In some aspects, the at least one inflow container and the at least one outflow container are syringes. The at least one inflow container may comprise at least 3 inflow syringes and the at least one outflow container may comprise at least 3 outflow syringes. The inflow syringes may be connected using a branch and the outflow syringes may be connected using a branch.

In some aspects, the method further includes connecting a catheter system to the at least one inflow container and the at least one outflow container through tubing, where the catheter system is operable to be deployed within the treatment region and isolate the treatment region.

In additional aspects, the method may further include connecting a motorized screw and/or at least one vacuum gauge to the at least one inflow container, the at least one outflow container, and/or the drawbar. The motorized screw may be controlled by an operator using a rheostat or may be controlled automatically. The vacuum gauges may be operable to ensure that a blockage within the isovolumetric pump system is addressed prior to it affecting treatment.

Additionally disclosed herein is a method of removing of a thrombus from a treatment region, including pumping an infusion fluid to the treatment region using an infusion pump of an isovolumetric pump system; suctioning fluid from the treatment region using a suction pump of the isovolumetric pump system; and adjusting a volume of fluid in the treatment region. Substantially the same quantity of fluid is suctioned from the treatment region and infused into the treatment region from the outflow container.

The treatment region is a region within a vessel containing a thrombus and the method may further include removing the thrombus from the treatment region. In some aspects, the method may further include connecting a catheter system to the infusion pump and the suction pump through tubing, where the catheter system is operable to be placed within the vessel and isolate the treatment region. The walls of the vessel neither distend nor collapse while internal volume is exchanged in the treatment region.

In some aspects, the method further includes monitoring pressure into and out of the treatment region using a pressure sensor connected to the infusion pump, the suction pump, and/or the controller. In further aspects, the method further includes automatically controlling the infusion pump and the suction pump, via the controller, to compensate for pressure in the treatment region based on the monitored pressure from the pressure sensor.

Additional embodiments and features are set forth in part in the description that follows, and will become apparent to those skilled in the art upon examination of the specification or may be learned by the practice of the disclosed subject matter. A further understanding of the nature and advantages of the disclosure may be realized by reference to the remaining portions of the specification and the drawings, which forms a part of this disclosure.

### BRIEF DESCRIPTION OF THE DRAWINGS

The description will be more fully understood with reference to the following figures and data graphs, which are presented as various embodiments of the disclosure and should not be construed as a complete recitation of the scope of the disclosure, wherein:

FIG. 1 is a diagram of an isovolumetric pump connection, with vasculature modeled as a pipe through which flow of an incompressible fluid is steady and laminar with no change in height along path of flow.

FIG. 2A is an isometric view of an example electronically automated isovolumetric pump with syringes.

FIG. 2B is an isometric view of an example electronically automated isovolumetric pump with syringes.

FIG. 3A shows an isovolumetric pump in one example.

FIG. 3B is a 3-way branch in one example.

FIG. 4 is a diagram of an isovolumetric pump mounted to a motorized screw in one example.

FIG. 5A shows one side of an example isovolumetric pump with a vacuum/pressure gauge and linear actuator.

FIG. 5B shows an isovolumetric pump in one example.

FIG. 6 is a diagram of an isovolumetric pump in one example.

FIG. 7A shows a breakout view of an isovolumetric pump in one example.

FIG. 7B shows an assembled view of the isovolumetric pump in FIG. 7A.

FIG. 8A shows a breakout view of an isovolumetric pump in one example.

FIG. 8B shows an assembled view of the isovolumetric pump in FIG. 8A.

FIG. 9 is a flowchart for a method of removing a thrombus in one example.

FIG. 10 is a flowchart for removing a thrombus from a treatment region in another example.

FIG. 11 shows an ex vivo testing apparatus included an insertion point for a balloon encapsulation and isovolumetric suction thrombectomy catheter through an on/off valve into a four-way branch in one example.

FIG. 12 provides a graphical representation for infusion without suction, suction without infusion, and isovolumetric infusion and suction.

### DETAILED DESCRIPTION

The disclosure may be understood by reference to the following detailed description, taken in conjunction with the drawings as described below. It is noted that, for purposes of illustrative clarity, certain elements in various drawings may not be drawn to scale. Several variations of the device are presented herein. It should be understood that various components, parts, and features of the different variations may be combined together and/or interchanged with one another, all of which are within the scope of the present application, even though not all variations and particular variations are shown in the drawings. It should also be understood that the mixing and matching of features, elements, and/or functions between various variations is expressly contemplated herein so that one of ordinary skill in the art would appreciate from this disclosure that the features, elements, and/or functions of one variation may be incorporated into another variation as appropriate, unless described otherwise.

Several definitions that apply throughout this disclosure will now be presented. As used herein, “about” refers to numeric values, including whole numbers, fractions, percentages, etc., whether or not explicitly indicated. The term “about” generally refers to a range of numerical values, for instance, ±0.5-1%, ±1-5% or ±5-10% of the recited value, that one would consider equivalent to the recited value, for example, having the same function or result.

The term “comprising” means “including, but not necessarily limited to”; it specifically indicates open-ended inclusion or membership in a so-described combination, group, series and the like. The terms “comprising” and “including” as used herein are inclusive and/or open-ended and do not exclude additional, unrecited elements or method processes. The term “consisting essentially of” is more limiting than “comprising” but not as restrictive as “consisting of.” Specifically, the term “consisting essentially of” limits membership to the specified materials or steps and those that do not materially affect the essential characteristics of the claimed invention. The terms “a,” “an,” and “the” are understood to encompass the plural as well as the singular. Thus, the term “a mixture thereof” also relates to “mixtures thereof.”

As used herein, the term “isovolumetric pump”, “pump”, or “isochoric actuator” means an external pump that is operable to facilitate adjustable isovolumetric infusion and suction within an endovascular catheter in the circulatory system.

Disclosed herein is a device and system for adjustable isovolumetric infusion and suction within an endovascular catheter in the circulatory system. In an embodiment, the device is a pump, such as an isovolumetric pump, for drawing and infusing the same volume of fluid from a sealed treatment region, thereby maintaining the same volume of fluid within the treatment region. In an embodiment, the isovolumetric pump ensures that the walls of the treatment region (e.g. vasculature) neither distend nor collapse while internal volume is exchanged.

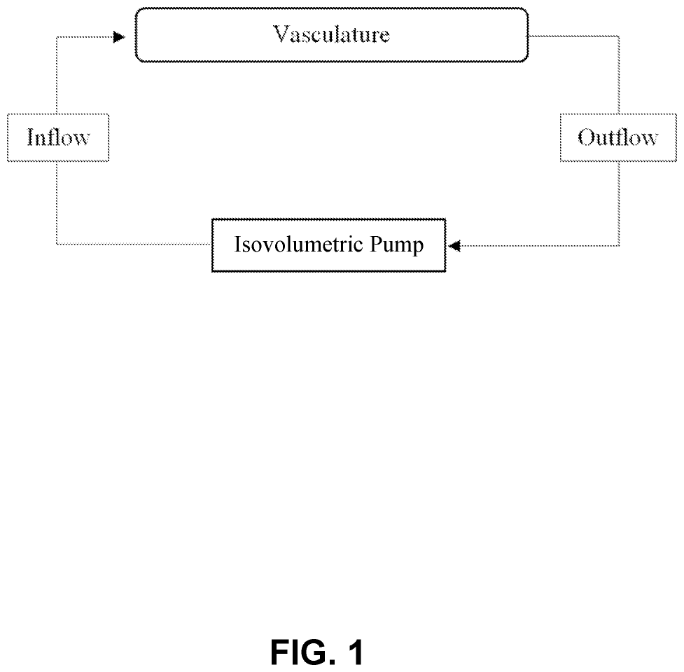

Prior to incorporation of the isovolumetric pump, vasculature is considered a sealed container. With an isovolumetric pump connection, vasculature is modeled as a pipe through which flow of an incompressible fluid is steady and laminar with no change in height along path of flow, as seen in FIG. 1.

Bernoulli equation may be applied to inflow and outflow connections to the vasculature, as seen in eqn. 1:

$${P}_{1} + \frac{1}{2} ρ {v}_{1}^{2} + ρ g {h}_{1} = {P}_{2} + \frac{1}{2} ρ {v}_{2}^{2} + ρ g {h}_{2} \qquad Eqn . 1$$

where P₁ is the pressure energy,

$$\frac{1}{2} ρ {v}_{1}^{2}$$

is the Kinetic energy per unit volume, and ρgh₁ is the potential energy per unit volume at the inlet of the vasculature, while

$${P}_{2} , \frac{1}{2} ρ {v}_{2}^{2} ,$$

and ρgh₂ represent the same properties at the outflow of the vasculature, respectively. ρ is the density of the fluid, v₁ and v₂ are the velocity of the fluid at the inlet and outlet of the vasculature, respectively, g is the acceleration of the fluid due to gravity, and h₁ and h₂ are the relative heights of the inlet and outlet of the vasculature, respectively. Additionally, the overall height of the circuit encompassing both the vasculature and the isovolumetric pump remains constantly level relative to itself.

For input and output containers at the same height, h₁=h₂, Δh is assumed to be 0:

$${P}_{1} + \frac{1}{2} ρ {v}_{1}^{2} = {P}_{2} + \frac{1}{2} ρ {v}_{2}^{2} \qquad Eqn . 2$$

As the isovolumetric pump constrains inflow to outflow and vice versa:

v₁=v₂ Eqn. 3

so that,

P₁=P₂ Eqn. 4

and at steady state the fluid passing in an out of an isovolumetric pump the pressure, velocities and flow rates will be identical at the inlet and outlet if there is no fluid loss.

Because of fluid loss in the system and elasticity of the tubing and vasculature, transients will arise in which the pressure and/or velocity must be modulated to maintain constant volume in the vasculature.

In an embodiment, the isovolumetric pump maintains a constant volume within an isolated treatment region in a patient while exchanging its internal volume. In some examples, the isovolumetric pump may be used to remove a thrombus from a treatment region by removing a volume from the treatment region while simultaneously exchanging the removed volume. The treatment region may be contained within or adjacent to a catheter for removal of large-volume venous thrombus in the extremities, chest, abdomen, and pelvis of a patient.

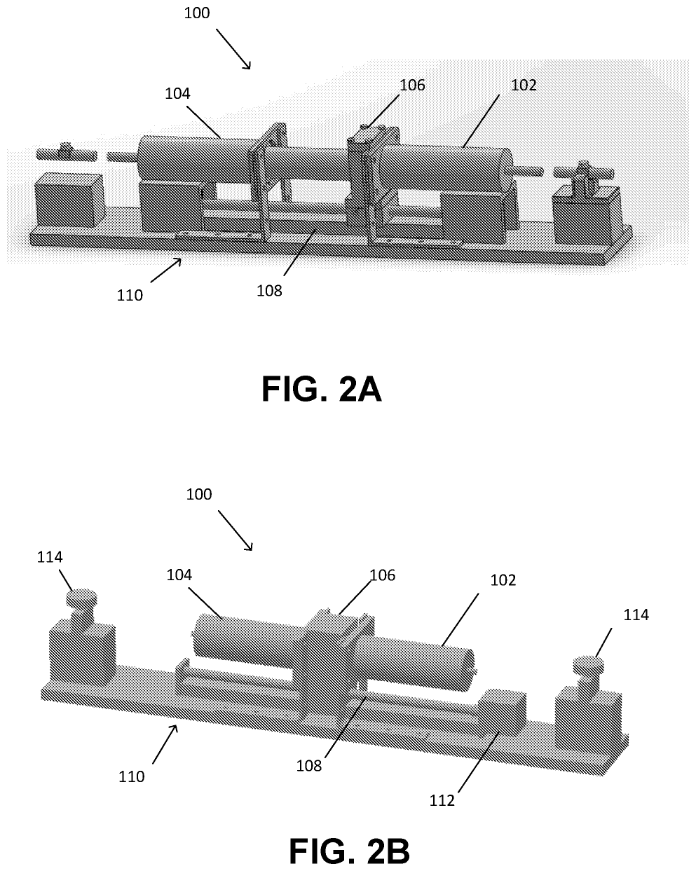

Referring to FIGS. 2A and 2B, the isovolumetric pump 100 may include at least one outflow container 102 connected to tubing coming from the treatment region in the patient and at least one inflow container 104 connected to tubing going to the treatment region in the patient. The isovolumetric pump 100 may further include a drawbar 106 between the outflow container 102 and the inflow container 104. The drawbar is operable to ensure the same quantity of fluid is drawn and injected from the inflow container and the outflow container. In various examples, the containers may be any size needed for exchange of volume within a treatment region. In at least one example, the inflow container and/or the outflow container is a syringe. In some examples, the syringe may be a 500 mL syringe. The isovolumetric pump may further include a linear actuator 108 with a motor 112 operable to moving the syringes linearly. The speed and direction of movement may be controlled by an operator. In some examples, the linear actuator 108 may be connected to the drawbar 106. The containers 102, 104, linear actuator 108, and/or drawbar 106 may be connected, directly or indirectly, to a mount 110. In some examples, the isovolumetric pump may further include one or more pressure gauges 114 for monitoring the pressure in one or both containers 102, 104. The pressure gauges 114 may be connected to the inflow container 104 and/or the outflow container 102 via tubing (not shown).

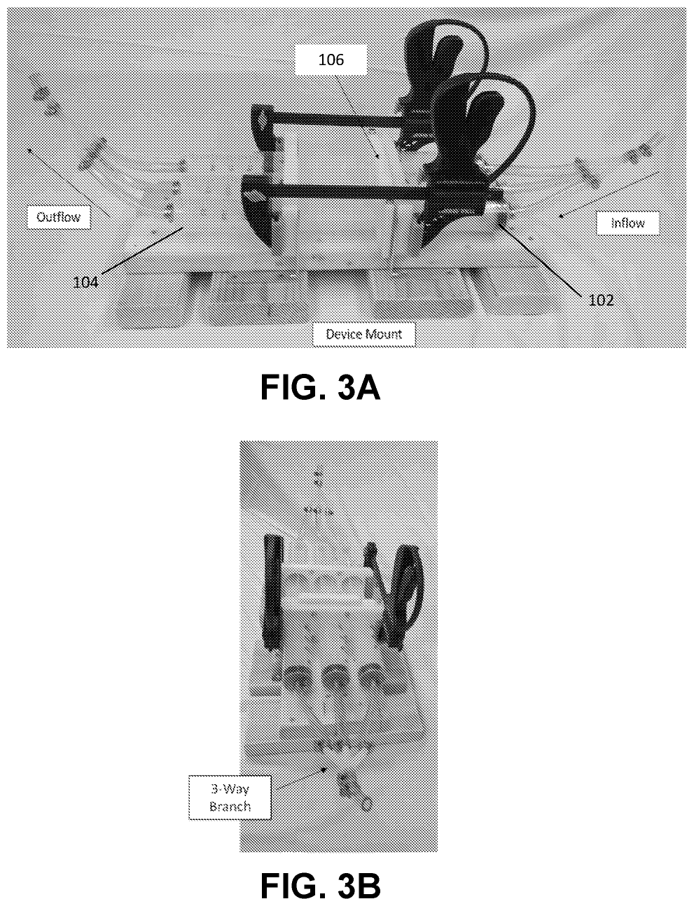

Referring to FIG. 3A, the isovolumetric pump may include at least one inflow syringe and at least one outflow syringe between a treatment region, where the volume of the inflow syringes matches the volume of the outflow syringes. The at least one inflow syringe and the at least one outflow syringe may be connected by a drawbar, such that as the drawbar is drawn back, the same quantity of fluid is drawn and injected from the treatment region. Any occlusion in the system may prevent further movement of the syringes, thus providing isochoric conditions within the treatment region while exchanging fluid volume. In various examples, the isovolumetric pump may include 1 inflow syringe and 1 outflow syringe, 2 inflow syringes and 2 outflow syringes, 3 inflow syringes and 3 outflow syringes, 4 inflow syringes and 4 outflow syringes, or 5 inflow syringes and 5 outflow syringes. In at least one example, as seen in FIG. 3A, the isovolumetric pump may include six total syringes, three inflow syringes for injecting fluid into the treatment region and three outflow syringes for withdrawing fluid from the treatment zone. The syringes may have a volume of 50 mL, 100 mL, 200 mL, 500 mL, or 1000 m L.

As seen in FIGS. 2A, 2B, 3A, and 3B, the syringes and/or linear actuator may be mounted on a syringe mount. In some examples, the inflow containers and/or the outflow containers may be connected to branches to combine the inflow or outflow volumes of multiple containers, respectively, as seen in FIG. 3B. Connections between the tubing, branches, and containers may include homogenous chemical bonding applied where possible to seal connection points. In addition, changes in the diameter through the isovolumetric pump may be minimized.

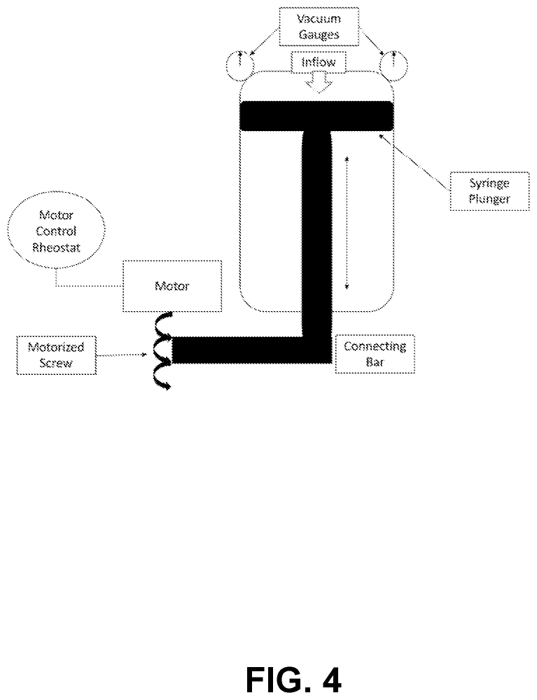

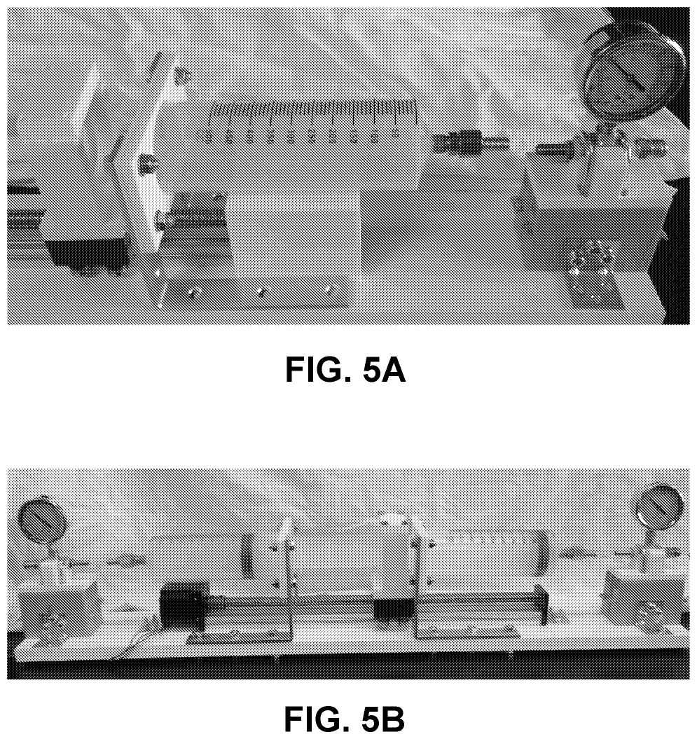

In another embodiment, the isovolumetric pump may further include one or more vacuum gauges, a motor, a motor control rheostat, and/or a motorized screw, as seen in FIG. 4. Vacuum gauges may be utilized to ensure that blockage within the system is addressed prior to it affecting treatment. FIGS. 5A and 5B show an isovolumetric pump with a vacuum/pressure gauge. The gauges may be monitored by the operator to avoid blockages in the system.

In some examples, the drawing and/or pushing of fluid into/from the syringe or multiple syringes may be accomplished using mounts to a motorized screw, as seen in FIG. 4. In other examples, the isovolumetric pump may include a linear actuator to draw or push fluid into/from the syringes, as seen in FIGS. 5A and 5B. In some examples, the linear actuator may move the drawbar linearly.

Referring to FIGS. 4, the isovolumetric pump may further include a motor control rheostat. In some examples, the motor may be controlled by an operator using the rheostat, with the flow rate and direction dependent upon its setting. In other examples, the motor may be controlled automatically. In some embodiments, the isovolumetric pump may further include a display and/or control buttons (e.g. start, stop, reverse, and/or reset buttons). The linear actuator may be run by an operator actively pressing a start/stop button. During travel, the speed of the electronic linear actuator may be adjusted using the motor speed rheostat, and the direction of travel may be reversed as desired by pressing the reverse button. The isovolumetric pump, by way of the linear actuator, may be bound within a specific length of travel, at which point the operator may press the reset button to operate the system in the opposite direction if desired. In this way, the cycling of fluid and recharging of the system may be electronically automated.

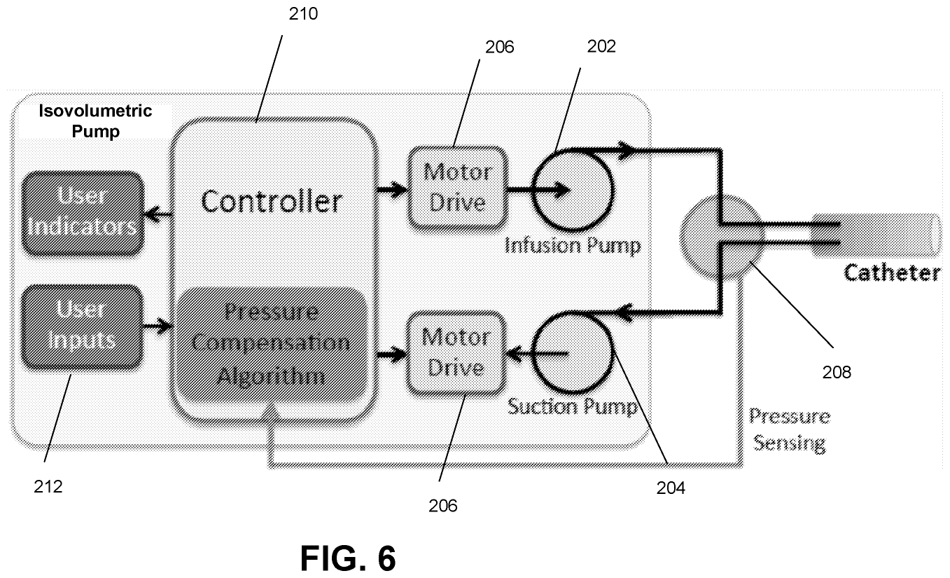

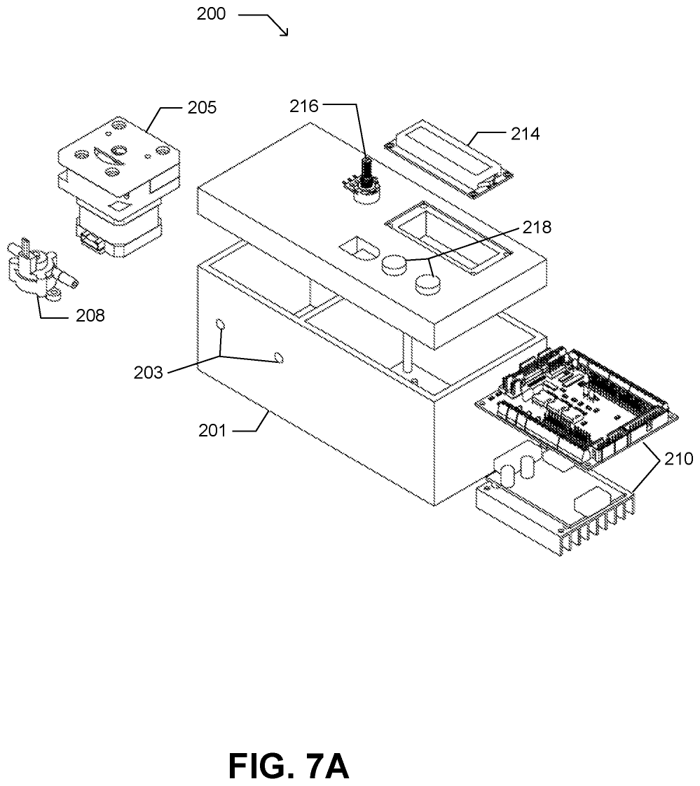

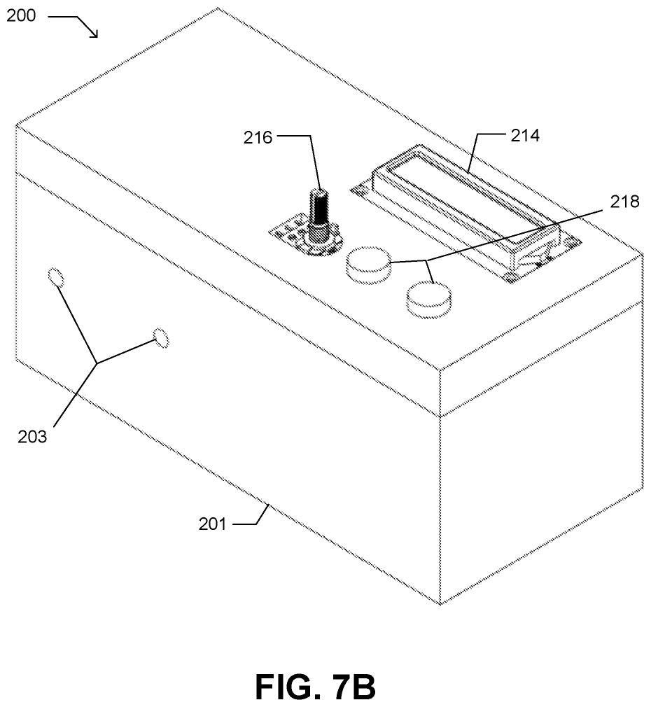

In another embodiment, the isovolumetric pump may include a low-complexity ergonomic design for infusion and suction of fluid, intuitive display/feedback of the device status, and small form factor to enhance ergonomic needs. In this embodiment, as seen in FIGS. 6, 7A, and 7B, the isovolumetric pump 200 may include an infusion pump 202 and a suction pump 204, one or more motor drives 206, a pressure sensor 208, a controller 210, and a user interface 212.

The one or more motor drives 206 may be operatively connected to or part of the infusion pump 202 or suction pump 204. The infusion pump 202 and the suction pump 204 may be operable to support differing infusion and suction flow rates. In some examples, the infusion pump 202 and/or the suction pump 204 may be a peristaltic pump, a continuous pump, or an intermittent pump. The infusion pump 202 may be operable to pump a fluid out of the isovolumetric pump 200 and into a treatment region, via a catheter. The infusion pump 202 may further be connected to an infusion source to provide the infusion fluid to the infusion pump. In some examples, the fluid to be infused is saline or any other biocompatible fluid. The suction pump 204 may be operable to suction fluid out of the treatment region, via the catheter, and into the isovolumetric pump 200. The suction pump 204 may further be connected to a reservoir for collecting the suctioned fluid.

The one or more motor drives 206 may be operable to provide variable speed and direction for the pumps. In some examples, the isovolumetric pump 200 includes two motor drives 206. In other examples, the isovolumetric pump includes a dual motor drive.

The pressure sensor 208 may be connected to the infusion pump 202 and/or the suction pump 204. In some examples, the pressure sensor 208 may be connected to the infusion pump 202 and the suction pump via the one or more motor drives 206. The pressure sensor 208 may further be connected to or in communication with a controller. In some examples, the pressure sensor 208 may be operable to achieve automatic compensation during real-time infusion and suction.

In some embodiments, the isovolumetric pump may further include a flow sensor connected to the infusion pump, the suction pump, and/or the controller. The flow sensor may be operable to monitor volumetric flow and/or fluid velocity into and out of the treatment region.

The controller 210 may be connected to the pressure sensor 208, the infusion pump 202, the suction pump 204, and the one or more motor drives 206. In some embodiments, the controller may be operable to control input and/or output pressures, volumetric flow rates, and/or fluid velocities from the suction pump and the infusion pump. In some examples, the controller 210 may further be operable to support a pressure compensation algorithm and dual motor drives. In an example, the controller may be a TB6560 controller. The controller 210 may further include at least one processor operable to carry out instructions for the pressure compensation algorithm. In an example, the processor may be an Arduino Uno shield motherboard. In an example, the pressure compensation algorithm receives the inflow and outflow pressures, determines a mismatch in the volume between inflow and outflow, and allows for compensation of volume to be added to the inflow or subtracted from the outflow.

Referring to FIGS. 7A and 7B, the infusion pump, suction pump, and motor drive may be combined as an infusion/suction pump with a dual motor drive 205. The components of the isovolumetric pump 200 may be contained within or on a housing 201. In some examples, the housing 201 may include one or more compartments for holding the infusion pump 202, the suction pump 204, one or more motor drives 206, the pressure sensor 208, and the controller 210. The housing 201 may further include two or more openings 203 operable for receiving tubing. The tubing may connect the infusion pump 202 and the suction pump 204 to a catheter forming a treatment region.

The user interface 212 may include a set of buttons and attachments to meet the end user anticipated needs. In some examples, the user interface 212 may include an LCD screen 214 or an analog screen, a speed knob switch 216, and/or an on/off switch 218. In other examples, the user interface includes screens, knobs, and/or power switches that interface with the housing wirelessly. For example, the controlling hardware and/or software may be housed in a device that connects wirelessly to the housing. In this example, the user interface may be within an application on a wireless device, such that the speed knob and on/off switch are not hardware but are controlled within an application on a device such as a phone, tablet, or laptop. In some examples, the LCD screen may be a display within the application on the wireless device. The LCD screen 214 or analog screen may be operable to display inflow and outflow flow rates. In some examples, the speed knob switch 216 may be a potentiometer. The speed knob switch may be connected to the one or more motor drives for controlling the pumping speed or volume of the infusion pump and/or the infusion pump.

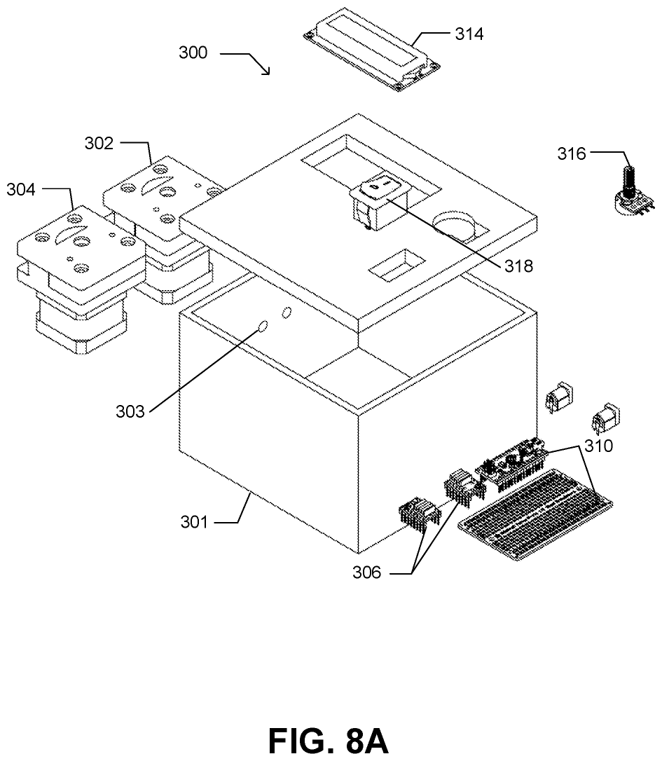

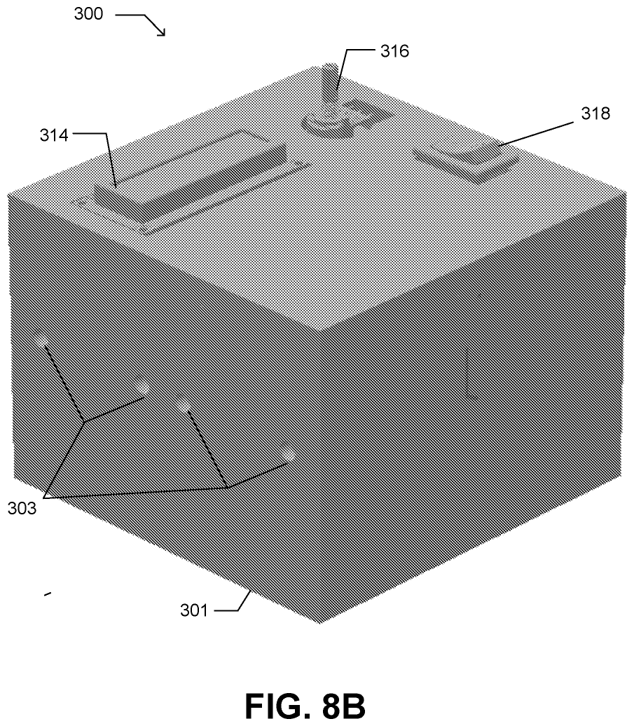

Referring to FIGS. 8A and 8B, in another embodiment, the isovolumetric pump 300 may include an infusion pump 302, a suction pump 304, two motor drives 306, a controller 310, and a user interface. In some embodiments, the isovolumetric pump 300 may further include a pressure sensor. The motor drives 306 may be operatively connected to the infusion pump 302 and the suction pump 304. The infusion pump 302 and the suction pump 304 may be operable to support differing infusion and suction flow rates. In some examples, the infusion pump 202 and/or the suction pump 204 may be a peristaltic pump, a continuous pump, or an intermittent pump. The infusion pump 302 may be operable to pump a fluid out of the isovolumetric pump 300 and into a treatment region, via a catheter. The infusion pump 302 may further be connected to an infusion source to provide the infusion fluid to the infusion pump. In some examples, the fluid to be infused is saline or any other biocompatible fluid. The suction pump 304 may be operable to suction fluid out of the treatment region, via the catheter, and into the isovolumetric pump 300. The suction pump 304 may further be connected to a reservoir for collecting the suctioned fluid.

The motor drive 306 may be connected to the infusion pump 302 and/or the suction pump 304. In some examples, the motor drive(s) 306 may be operable to provide variable speed and direction for the pumps. In some examples, the isovolumetric pump 300 includes two motor drives 206. In other examples, the isovolumetric pump includes a dual motor drive.

The controller 310 may be operable to support a pressure compensation algorithm and dual motor drives. In an example, the controller may be a TB6560 controller. The controller 310 may further include at least one processor operable to carry out instructions for pressure compensation. In an example, the processor may be an Arduino Uno shield motherboard. In some embodiments, the controller may be operable to control input and/or output pressures, volumetric flow rates, and/or fluid velocities from the suction pump and the infusion pump.

In some embodiments, the isovolumetric pump may further include a flow sensor connected to the infusion pump, the suction pump, and/or the controller. The flow sensor may be operable to monitor volumetric flow and/or fluid velocity into and out of the treatment region.

The components of the isovolumetric pump 300 may be contained within or on a housing 301. In some examples, the housing 301 may include a compartment for holding the infusion pump 302, the suction pump 304, the motor drive(s) 306, and the controller 310. The housing 301 may further include two or more openings 303 operable for receiving tubing. The tubing may pass through the openings 303 to connect the infusion pump 302 and the suction pump 304 to a catheter forming a treatment region. The tubing may also pass through the openings 303 to connect the infusion pump 302 to the infusion fluid source and connect the suction pump 304 to the reservoir.

The user interface 312 may include a set of buttons and attachments to meet the end user anticipated needs. In some examples, the user interface 312 may include an LCD screen 314 or an analog screen, a speed knob switch 316, and/or an on/off switch 318. In other examples, the user interface includes screens, knobs, and/or power switches that interface with the housing wirelessly. For example, the controlling hardware and/or software may be housed in a device that connects wirelessly to the housing. In this example, the user interface may be within an application on a wireless device, such that the speed knob and on/off switch are not hardware but are controlled within an application on a device such as a phone, tablet, or laptop. In some examples, the LCD screen may be a display within the application on the wireless device. The LCD screen 314 or analog screen may be operable to display inflow and outflow flow rates. In some examples, the speed knob switch 316 may be a potentiometer.

The base, drawbar, branches, syringe mounts, and/or housing may be composed of plastic such as, but not limited to, polyethylene terephthalate (PETE), high-density polyethylene (HDPE), polyvinyl chloride (PVC), polypropylene (PP), and polystyrene (PS). In some examples, the drawbar, branches, syringe mounts, or housing may include polylactic acid. In at least some examples, the drawbar, branches, syringe mounts, or housing may be 3D printed. In some examples, the base of the device may be made of wood or plastic and 90-degree plate metal bends.

In an embodiment, an isovolumetric system may include an isovolumetric pump and a catheter or catheter system that is operable to form the treatment region for isovolumetric infusion and suction. In some examples, the isovolumetric pump may be connected to a catheter system deployed within the treatment region to assist in the removal of a thrombus from the treatment region. The inflow/infusion tubing and the outflow/suction tubing may be connected to one or more lumen in the catheter system. For example, the catheter system may isolate a region of a vessel (e.g. using a two-balloon system), creating the treatment region, and the isovolumetric pump may be connected to the catheter such that it may withdraw fluid from the isolated treatment region, including the thrombus, through the catheter and return an equal volume of fluid through the catheter to the treatment region. The catheter may include separate lumen for the infusion of infusion and suction of fluid within the treatment region by the isovolumetric pump.

Infusion from the isovolumetric pump into the treatment region of the catheter system placed in a vein may cause a gradual increase in venous pressure and diameter changes. These changes may be reduced by >95% with isovolumetric infusion and suction. Additionally, isovolumetric flushing with the isovolumetric pump may lead to <5% residual thrombus in the treatment region.

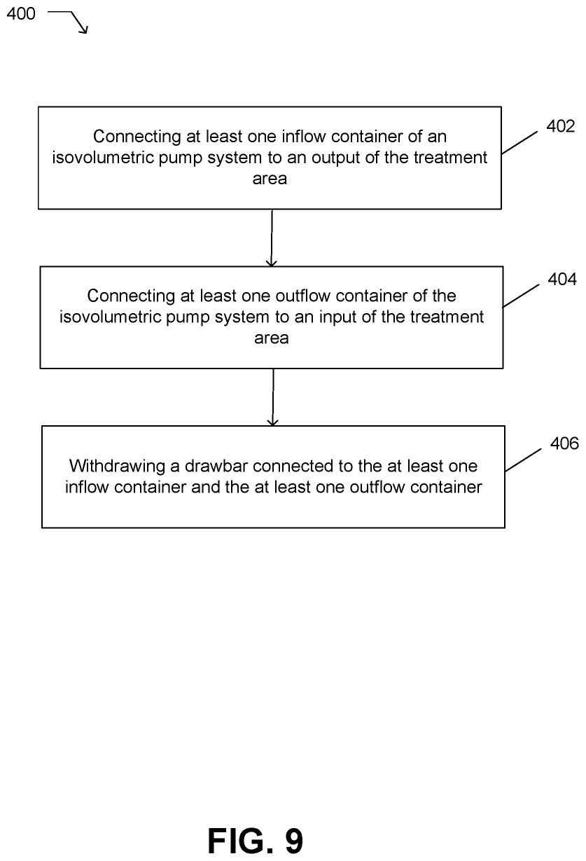

Further provided herein are methods of removing of a thrombus from a treatment region. Referring to FIG. 9, a flowchart is presented in accordance with an example embodiment. The method 400 is provided by way of example, as there are a variety of ways to carry out the method. The method 400 described below can be carried out using the configurations illustrated in FIGS. 2A-5B, for example, and various elements of these figures are referenced in explaining example method 400. Each block shown in FIG. 9 represents one or more processes, methods or subroutines, carried out in the example method 400. Furthermore, the illustrated order of blocks is illustrative only and the order of the blocks can change according to the present disclosure. Additional blocks may be added or fewer blocks may be utilized, without departing from this disclosure.

The example method 400 is a method of removing of a thrombus from a treatment region. The treatment region may be a region within a vessel containing a thrombus. The treatment region may be a region within a vessel containing a thrombus, such as a thrombosed/occluded arterial segment. For example, the method may be used for an arterial thrombectomy or atherectomy. The example method 400 can begin at block 402. At block 402, at least one inflow container of an isovolumetric pump system may be connected to an output of the treatment region. In some examples, the inflow container may be a syringe. The output of the treatment region may be an output of a catheter system inserted into a vessel of a patient to form the treatment region.

At block 404, at least one outflow container of the isovolumetric pump system may be connected to an input of the treatment region. In some examples, the outflow container may be a syringe. The input of the treatment region may be an input of a catheter system inserted into a vessel of a patient to form the treatment region.

At block 406, a drawbar connected to the at least one inflow container and the at least one outflow container may be withdrawn. The drawbar is connected to both containers such that the same quantity of fluid is drawn from the treatment region into the inflow container and injected into the treatment region from the outflow container.

The method 400 may optionally include pulling fluid and the thrombus from the treatment region into the at least one inflow container and simultaneously pushing fluid from the at least one outflow container into the treatment region. The walls of the vessel neither distend nor collapse while internal volume is exchanged in the treatment region.

The method 400 may additionally include connecting a catheter system to the at least one inflow container and the at least one outflow container through tubing. The catheter system may be operable to be deployed within the treatment region and isolate the treatment region.

The method 400 may also include connecting a motorized screw and/or a vacuum gauge to the at least one inflow container, the at least one outflow container, and/or the draw-bar. In some examples, the motorized screw may be controlled by an operator using a rheostat or may be controlled automatically. In additional examples, the vacuum gauges may be operable to ensure that a blockage within the isovolumetric pump system is addressed prior to it affecting treatment.

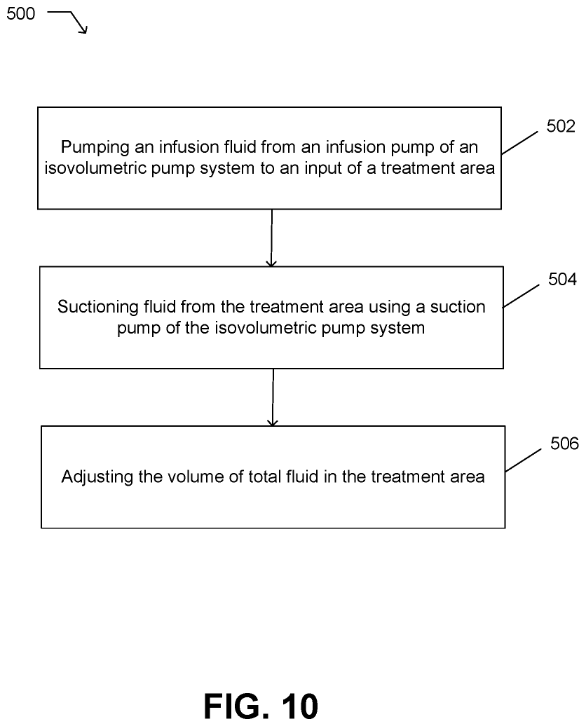

Further provided herein are additional methods of removing of a thrombus from a treatment region. Referring to FIG. 10, a flowchart is presented in accordance with an example embodiment. The method 500 is provided by way of example, as there are a variety of ways to carry out the method. The method 500 described below can be carried out using the configurations illustrated in FIGS. 6-8B, for example, and various elements of these figures are referenced in explaining example method 500. Each block shown in FIG. 10 represents one or more processes, methods or subroutines, carried out in the example method 500. Furthermore, the illustrated order of blocks is illustrative only and the order of the blocks can change according to the present disclosure. Additional blocks may be added or fewer blocks may be utilized, without departing from this disclosure.

The example method 500 is a method of removing of a thrombus from a treatment region. The treatment region may be a region within a vessel containing a thrombus, such as a thrombosed/occluded arterial segment. For example, the method may be used for an arterial thrombectomy or atherectomy. The example method 500 can begin at block 502. At block 502, an infusion fluid may be pumped from an infusion pump of an isovolumetric pump system to an input of the treatment region. The input of the treatment region may be an input of a catheter system inserted into a vessel of a patient to form the treatment region. The catheter system may be operable to be deployed within the treatment region and isolate the treatment region. The infusion pump may be connected to the catheter system via tubing. The infusion fluid may be supplied to the infusion pump by an infusion fluid source also connected to the infusion pump. In some examples, the infusion fluid may be saline.

At block 504, fluid may be suctioned from the treatment region using a suction pump of the isovolumetric pump system. The output of the treatment region may be an output of a catheter system inserted into a vessel of a patient to form the treatment region. The suction pump may be connected to the catheter system via tubing. In some examples, the suctioned fluid may be received in a reservoir that is also connected to the suction pump to collect fluid removed from the treatment region.

At block 506, a volume of total fluid in the treatment region may be adjusted by adjusting the infusion into or suction out of the treatment region using one or more motor drives connected to the infusion pump and the suction pump. The one or more motor drives allow the infusion pump and the suction pump to add and remove fluid from the treatment region such that the same quantity of fluid is suctioned from the treatment region by the suction pump and infused into the treatment region from the infusion pump. In some examples, the one or more motor drives may be controlled by a controller. The walls of the vessel neither distend nor collapse while internal volume is exchanged in the treatment region. The volume of fluid pumped into and out of the treatment region may be substantially similar. In some examples, the volumes may be within about 1% to about 20% of each other.

The method 500 may also include monitoring the pressure of the fluids being pumped into and/or out of the treatment region using a pressure sensor and/or a flow sensor connected to the infusion pump and/or suction pump. In some examples, the pressure sensor may be connected to the controller that is operable to operable to provide pressure compensation and operate the one or more motor drives. In other examples, the method may include monitoring volumetric flow and/or fluid velocity into and out of the treatment region using a flow sensor connected to the infusion pump, the suction pump, and/or the controller.

The method 500 may optionally include monitoring the geometry of the vessel in the treatment region using modalities such as MRI, ultrasound, or fluoroscopy to measure vessel wall dimensions and/or volume within the treatment region.

### Examples

### Example 1: Ex Vivo Benchtop Testing Apparatus

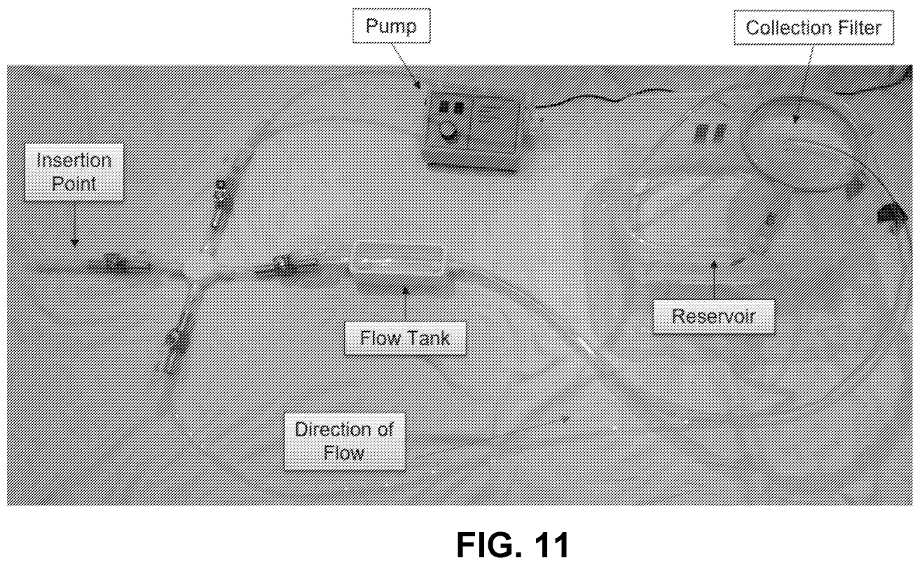

An ex vivo testing apparatus included an insertion point for a balloon encapsulation and isovolumetric suction thrombectomy catheter through an on/off valve into a four-way branch, as seen in FIG. 11. The catheter is an adjustable catheter system with isovolumetric suction and restoration of fluid for the removal of a thrombus and includes two encapsulation balloons, an inner catheter, an outer sheath surrounding at least a portion of the inner catheter, and an optional agitator.

Each branch was connected to an on/off valve, with a first branch allowing fluid inflow from the pump, which drew fluid from a reservoir. A second branch of the four-way branch led to the same reservoir, completing the primary circuit of flow. The final branch led to a flow tank, in which was placed a “clotsicle”. The outflow of this flow tank led to the collection filter, in which was placed a filter to collect thrombus released from the clotsicle. The collected thrombus was massed and compared to pre and post-massed clotsicles in order to quantify the thrombus quantity which escaped downstream of the treatment region, as compared to that collected by the isovolumetric pump.

Clotsicles included sections of clear surgical tubing into which porcine blood was poured and allowed to rest over the course of 24-48 hours around central shafts to form hollow clots which allow for flow. The clotsicles were massed before and after benchtop test to determine quantity of the thrombus removed and allow for the quantifiable comparison of the thrombus mass collected by the isovolumetric pump compared to that remaining and that which traveled downstream of treatment region.

### Example 2: In Vivo Isovolumetric Pump Applied to Porcine Thrombosis

The isovolumetric pump was connected to a balloon encapsulation and isovolumetric suction thrombectomy catheter which was deployed in a position isolating a thrombus in a pig's inferior vena cava. The porcine thrombus was drawn into the inflow of one of the syringes of the isovolumetric pump, through an on/off valve which allowed for the flow circuit to remain closed while the isovolumetric pump was connected and disconnected.

Porcine thrombuses were collected from two separate pigs in two separate surgeries in-tandem with the balloon encapsulation and isovolumetric suction thrombectomy catheter.

The isovolumetric pump drew a porcine thrombus through the inlet of one of its syringes while infusing the same volume of sterile saline back into the isolated surgical treatment zone.

### Example 3: Optimization of Isovolumetric Infusion and Suction

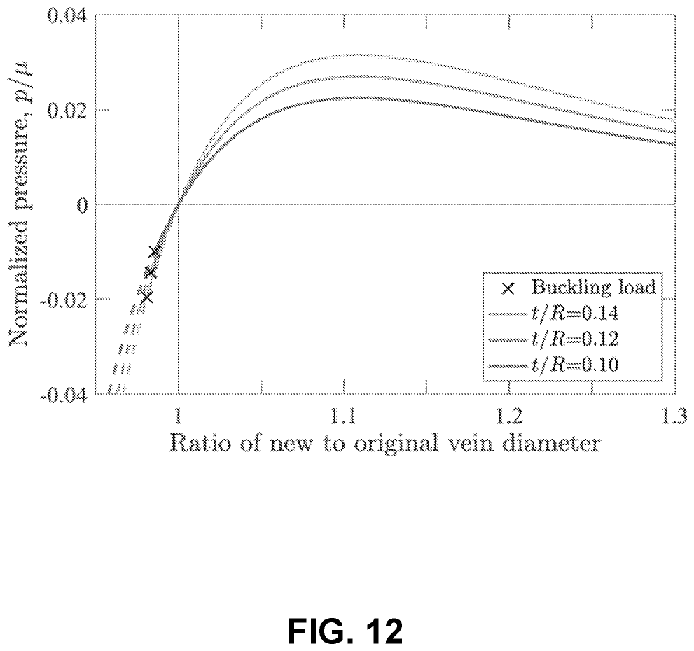

Using continuum mechanics methods now standard in vascular biomechanics, a thin region of vein within a treatment region was predicted to respond to either a positive pressure (p) arising from over-inflation or a negative pressure arising from aspiration. Because pressure is the variable most accurately measured by the isovolumetric pump, the baseline model asked how wall distension varied with pressure. For demonstration, a hyperelastic material model with two parameters are plotted in FIG. 12 (high strain shear modulus μ≈0.5−2.5 MPa, small strain modulus 0.3 MPa, and Poisson ratio, v=0.5). Note that unlike arteries, the longitudinal and transverse moduli of a vein are not different statistically. Using geometric relationships and assumptions, namely that the vein is incompressible, in conjunction with this constitutive law and equations of equilibrium, a final expression for the pressure versus vessel diameter was then derived for positive and negative pressure as p/μ=−λ_{θ}⁻⁷(λ_{θ}⁻⁶−1)(t/R), in which λ_{θ} is the circumferential stretch (current circumference divided by unstretched circumference). For negative pressure (suction), this expression was valid only up to the radial buckling pressure: p/μ=−(t/R)² for an incompressible vein wall.

Additionally, the change in vessel radius due to pressure was evaluated using an ex vivo flow system (FIG. 11). A portion of inferior vena cava was spliced into the ex vivo flow set up. A balloon encapsulation and isovolumetric suction thrombectomy catheter was advanced over the wire into the middle of an ex vivo vena cava segment. Physiologic saline was perfused through the circuit at 25 mL/s using the isovolumetric pump to provide isovolumetric suction and infusion through the catheter. The suction was performed at 10 kPa, 15 kPa, and 20 kPa. This was performed with and without concurrent isovolumetric infusion. The diameter of the vessel was measured before and during the suction with and without infusion. The change in vein diameter was then quantified.

The final expression of the vessel size versus pressure that was found using the Law of Laplace was

$$P = \frac{v ⋆ T}{R} {λ}_{θ}^{- 7} ⋆ ( {λ}_{θ}^{- 6} - 1 )$$

where v is the Poisson Ratio of the vena cava, T is the initial thickness of the vessel, R is the initial radius of the vessel, and) e is the unit change in radius of the vessel. FIG. 12 provides a graphical representation of this equation for infusion without suction, suction without infusion, and isovolumetric infusion and suction. All flow experiments were performed in triplicates, and non-parametric one-way ANOVA was used to evaluate differences between suction parameters.

Under physiological loading (p˜1 kPa, much lower than arterial pressure, so that p/μ<0.01) the vein inflates with increasing pressure (FIG. 12). For evacuation of the treatment region faster than inflation of it, suction occurs; too much suction causes collapse by buckling (X's in FIG. 12). Inflation with insufficient evacuation leads to bulging, as in the inflation of a party balloon. To ensure patient safety, isovolumic infusion will focus within the safe zone. Absent isovolumic pumping, average vein diameter change was 22.3% at 10 kPa, 30.7% at 15 kPa, and 33.3% at 20 kPa applied through the catheter. Isovolumetric infusion and suction performed through the catheter brought this into the safe zone. A diameter change of 2.3% at 10 kPa, 1.4% at 15 kPa, and 1.8% at 20 kPa (p<0.001) was observed with a first catheter prototype. Preliminary tests with a second catheter prototype suggest even less vein diameter change at 0.96% (p=0.003). Vein wall histological analysis showed no evidence of media fissures or elastic lamina breaks. This example demonstrates feasibility of the isovolumetric pump to preserve vein wall diameters.

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the presently disclosed embodiments teach by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. An isovolumetric pump system for removal of a thrombus from a treatment region, comprising:
an infusion pump connected to an input of the treatment region, the infusion pump operable to pump an infusion fluid into the treatment region;
a suction pump connected to an output of the treatment region, the suction pump operable to pump fluid out of the treatment region;
at least one motor drive operatively connected to the infusion pump and the suction pump; and
a controller operatively connected to the infusion pump and the suction pump via the at least one motor drive,
wherein the controller is operable to ensure that substantially the same volume of fluid is maintained within the treatment region by controlling input and/or output pressures, volumetric flow rates, and/or fluid velocities from the suction pump and the infusion pump.

2. The isovolumetric pump system of claim 1, wherein the treatment region is a region within a vessel containing a thrombus.

3. The isovolumetric pump system of claim 2, further comprising a catheter system operable to be placed within the vessel and isolate the treatment region.

4. The isovolumetric pump system of claim 3, wherein the catheter system is connected to the infusion pump and the suction pump through tubing.

5. The isovolumetric pump system of claim 2, wherein the isovolumetric pump system is operable to remove the thrombus from the treatment region.

6. The isovolumetric pump system of claim 5, wherein the isovolumetric pump system is operable to ensure that walls of the vessel neither distend nor collapse while internal volume is exchanged in the treatment region.

7. The isovolumetric pump system of claim 1, further comprising a pressure sensor connected to the infusion pump, the suction pump, and/or the controller.

8. The isovolumetric pump system of claim 7, wherein the pressure sensor and the controller are operable to perform pressure compensation within the treatment region and control the infusion pump and the suction pump automatically.

9. The isovolumetric pump system of claim 1, further comprising a flow sensor connected to the infusion pump, the suction pump, and/or the controller, the flow sensor operable to monitor volumetric flow and/or fluid velocity into and out of the treatment region.

10. The isovolumetric pump system of claim 1, wherein the at least one motor drive is a dual motor drive.

11. The isovolumetric pump system of claim 1 further comprising a housing comprising:
at least one compartment for containing the infusion pump, the suction pump, the at least one motor drive, and the controller;
two or more openings on a side of the housing operable for receiving tubing connected to the infusion pump and the suction pump; and
an outer surface operable for supporting a user interface.

12. The isovolumetric pump system of claim 11, wherein the user interface comprises:
an LCD screen connected to the controller and operable for displaying inflow and outflow rates from the infusion pump and the suction pump;
a speed knob switch operatively connected to the at least one motor drive; and
an on/off switch.

13. A method of removing of a thrombus from a treatment region comprising:
pumping an infusion fluid to the treatment region using an infusion pump of an isovolumetric pump system;
suctioning fluid from the treatment region using a suction pump of the isovolumetric pump system; and
adjusting a volume of fluid in the treatment region,
wherein substantially the same volume of fluid is suctioned from the treatment region and infused into the treatment region.

14. The method of claim 13, wherein the treatment region is a region within a vessel containing a thrombus.

15. The method of claim 14, further comprising connecting a catheter system to the infusion pump and the suction pump through tubing, wherein the catheter system is operable to be placed within the vessel and isolate the treatment region.

16. The method of claim 14, further comprising removing the thrombus from the treatment region.

17. The method claim 14, wherein walls of the vessel neither distend nor collapse while internal volume is exchanged in the treatment region.

18. The method of claim 13, further comprising monitoring pressure into and out of the treatment region using a pressure sensor connected to the infusion pump, the suction pump, and/or a controller.

19. The method of claim 18, further comprising automatically controlling the infusion pump and the suction pump, via the controller, to compensate for pressure in the treatment region based on the monitored pressure from the pressure sensor.

20. The method of claim 1, further comprising monitoring volumetric flow and/or fluid velocity into and out of the treatment region using a flow sensor connected to the infusion pump, the suction pump, and/or the controller.
